(19) United States
(12) Patent Application Publication
(10) Pub. No.: US 2018/0028698 A1
(43) Pub. Date: Feb. 1, 2018

(54) Alternate Fast, Convenient and Efficient Device to Replace the Use of Prep Pad Soaked With a Variety of Solutions

(71) Applicant: Hirouye Teshome, Pittsburgh, PA (US)

(72) Inventor: Hirouye Teshome, Pittsburgh, PA (US)

(73) Assignee: Hirouye Teshome, Pittsburgh, PA (US)

(21) Appl. No.: 15/728,235

(22) Filed: Oct. 9, 2017

Related U.S. Application Data
(62) Division of application No. 15/017,566, filed on Feb. 5, 2016.

(51) Int. Cl. A61L 2/00; A47L 13/17; A61L 2/232; G02C 13/00

(52) U.S. Cl. CPC A61L 2/0082; G02C 13/00; A47L 13/17; A61L 2/232; B65D 2583/045

(57) ABSTRACT

When using a Prep Pad that contains a solution soaked with alcohol, adhesive or patch remover, or any other kind of antibacterial agent, the act of operating it while trivial for one time use can quickly become inefficient, mundane and time consuming. The invention packs multiple, but single use encapsulated Pellets inside a cylindrical refill apparatus. The Pellet is made of soft material and it may be soaked or un-soaked. The refill apparatus is then inserted into a cylindrical pen like structure that automatically peels the Pellet when operated. The result is that in a few pen like clicks, an operator, using a single hand, can quickly get ready to cleanse or apply a Pellet onto a surface. An operator can easily store capacity for multiple applications in a pen pocket. The single hand use, quickness to readiness and compact easy storage makes the invention ideal alternative to a Prep Pad like use.

### BACKGROUND OF THE INVENTION

Prep Pad's are commonly used to clean, remove or kill bacteria from a skin prior to injection or incision. They are also used for cleaning in non-living applications. Their use typically involves tearing a Prep Pad with fingers, taking out an antibacterial soaked material (like alcohol for example) and rubbing it onto a surface. The routine step of tearing, fishing for the antibacterial soaked material and applying it to a surface can quickly become inefficient, mundane and time consuming if one has to do it repetitively. Plus it typically requires use of two hands. It is not compact enough to carry around either. For example, a Diabetic who has to prick his or her finger multiple times a day and then finds a preferably sterile cotton to wipe off the first drop of blood will quickly find out that this is yet another inefficient step in the work load of Diabetes management. Removing any step of inefficiency is also a motivating factor as a father of a Type 1 Diabetic son. A nurse or a practitioner who has a long line of flu vaccine recipients will also have to go through the same repetitive task, often times wearing cumbersome latex gloves. Even cleaning hard to reach electronics or external device that may later be attached to a body can get tricky with a Prep Pad. Please see examples and varieties of Prep Pad in FIG. 1.

### BRIEF SUMMARY OF THE INVENTION

The invention allows fast and efficient alternative to the use of Prep Pad. In addition it gives the option of dry swiping the cleaned surface.

The invention is composed of four main parts. An outer cylindrical pen like structure, a peeling mechanism, an inner cylindrical refill tube that houses one or more Pellet and a refill Pellet. A Pellet may or may not be encapsulated. For example, a Pellet may be soaked with a cleansing antibacterial, adhesive or adhesive remover solution and then encapsulated. Or a Pellet may just be an encapsulated soft material that is dry but sterile. A Pellet may also be a soft material that is dry and not encapsulated.

In a couple of pen like clicks the invention allows an operator to be ready to clean a surface. Whether it to be to prepare for an injection, incision or some other purpose. In addition the invention gives better control in cleaning. For a Diabetic that typically has to wipe the first drop of blood, dry swiping using cotton like material is also clicks away. A signature benefit of the invention is that it allows a single hand use. An operator can peel and apply it to a surface using a single hand.

### DEFINITIONS FOR WORDS IN CLAIMS

### Planar Object

A 2-Dimensional material, when unfolded has the thickness of no more than a 2 mm. In addition, it's thickness to perimeter ratio is less than 5%.

### Non-Planar Object

A 3-Dimensional (3-D) material that is 3-D in the ordinary sense of 3-D. Additionally it is NOT a Planar Object. A geometry constructed with redundancy of Planar Object (including but not limited to rolled or multiple times folded Planar Object) is considered a Non-Planar Object.

### Pellet

A Non-Planar Object, absorbing or non-absorbing material that may be soaked or un-soaked to contain a solution that includes but not limited to antibacterial, antiseptic, cleansing agent, adhesive or adhesive remover. A Pellet may be wrapped, encapsulated or not capsulated.

### Prep Pad

A Planar Object that is typically soaked or un-soaked with a variety of solutions including but not limited to alcohol, antibacterial agents, adhesive, adhesive remover or any combination thereof. It is typically packaged before use. Please see examples and varieties in FIG. 1.

### Manual Peeling

An act or operation using bare hands and fingers to tear open a Prep Pad. Using a pair of scissors or a knife with one hand and a Prep Pad with the other hand is also considered Manual Peeling.

### Automatic Peeling

An automatic way of peeling as understood in the ordinary meaning of the word automatic. In addition, an act of peeling or tearing in a manner that is not Manual Peeling.

### BRIEF DESCRIPTION OF DRAWINGS

FIG. 1A shows a typical sterile Prep Pad soaked in 70% Isopropyl Alcohol, a common antibacterial or antiseptic solution.

FIG. 1B shows a sterile Prep Pad soaked in 70% Isopropyl Alcohol and also has a solution with adhesive characteristic as the alcohol dries up.

FIG. 1C shows a Prep Pad soaked in an adhesive remover solution.

FIG. 1D shows a Prep Pad soaked in a lens cleaning solution that can be used to clean eye glasses or camera lenses.

FIG. 2A is a cross sectional side view of one example of a complete apparatus that has an outer cylindrical body with a pushing lever, two inner refill cylindrical bodies stacked with encapsulated Pellets, a screw on peeling mechanism on each end and a cap.

FIG. 2B is an outer side view showing the push lever, laddered slide for the push lever, screw on peeling mechanism, its cap and a pen pocket clip.

FIG. 2C is an outer side view from the opposite end of FIG. 2B.

FIG. 2D is a cross sectional side view of the sample outer cylindrical body without the refills and the screw on peeling mechanisms.

FIG. 2E is a cross sectional side view of the left peeling mechanism.

FIG. 2F is a cross sectional side view of the right peeling mechanism.

FIG. 3 is a cross sectional side view of an outer cylindrical body with a pincer like pushing mechanism.

FIG. 3 is a cross sectional side view of an outer cylindrical body with a pincer like pushing mechanism.

FIG. 4A is a cross sectional side view of an outer cylindrical body with one of two rotating pushing mechanism.

FIG. 4B is a 90 degree cross sectional side view of FIG. 4A.

FIG. 4C is a magnified cross sectional side view of the first of two rotating pushing mechanism.

FIG. 5A is a cross sectional side view of an outer cylindrical body with the second of two rotating pushing mechanism. Please note the 90 degree cross sectional side view of FIG. 5A is similar to FIG. 4B with a push rod that is solid at the center but with the same triangular edge.

FIG. 5B is magnified cross sectional side view of the second of two rotating pushing mechanism.

FIG. 5C is a magnified 90 degree cross sectional side view of FIG. 5B.

FIG. 6A is a cross sectional side view of a peeling mechanism.

FIG. 6B, FIG. 6C and FIG. 6D is a cross sectional 90 degree side view of a peeling tip in FIG. 6A.

FIG. 7 is a cross sectional side view of a sample inner cylindrical or hollow body refill. It may or may not have a slot on the side.

FIG. 8 is an additional example of FIG. 7. that may or may not be spring loaded.

FIG. 9A is a cross sectional side view of a sample Pellet refill.

FIG. 9B shows a bottom tip of a Pellet that may or may not be perforated.

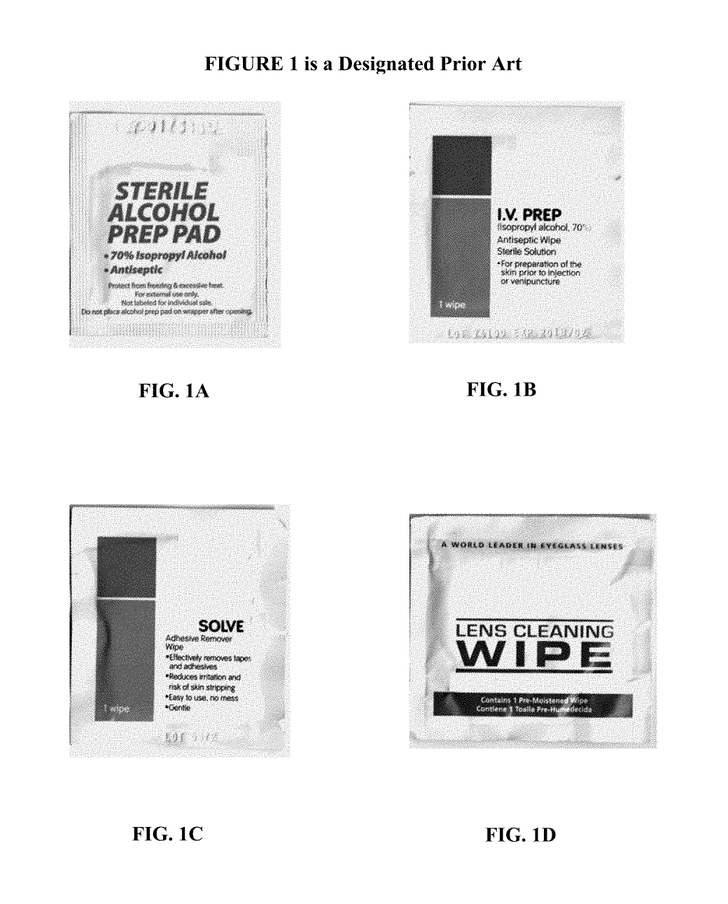

### DETAILED DESCRIPTION OF THE INVENTION

The typical process in preparing a body surface prior to injection involves pulling an alcohol soaked Prep Pad from a box which typically comes in pairs. Tearing one off and then tearing the single Prep Pad further typically using two hands to expose a soaked planar disinfectant and finally applying it as needed to cleanse an intended area. This process can quickly become inefficient, mundane and time consuming when it's done repetitively.

A similar process is used to clean an area when a Diabetic pricks a finger, attaches a sensor or an inset for a pump. An electronic sensor is also cleaned in a similar manner before it is attached to the body.

In the beauty industry too, cleaning nails, removing nail polish or makeup may involve similar processes.

Cleaning electronics, lenses or camera may also fall into the same process.

The invention improves the process of manually pealing a Prep Pad by utilizing a pen like structure that simply performs multiple clicks to get ready to cleanse or disinfect a surface. And it does this typically using only one hand.

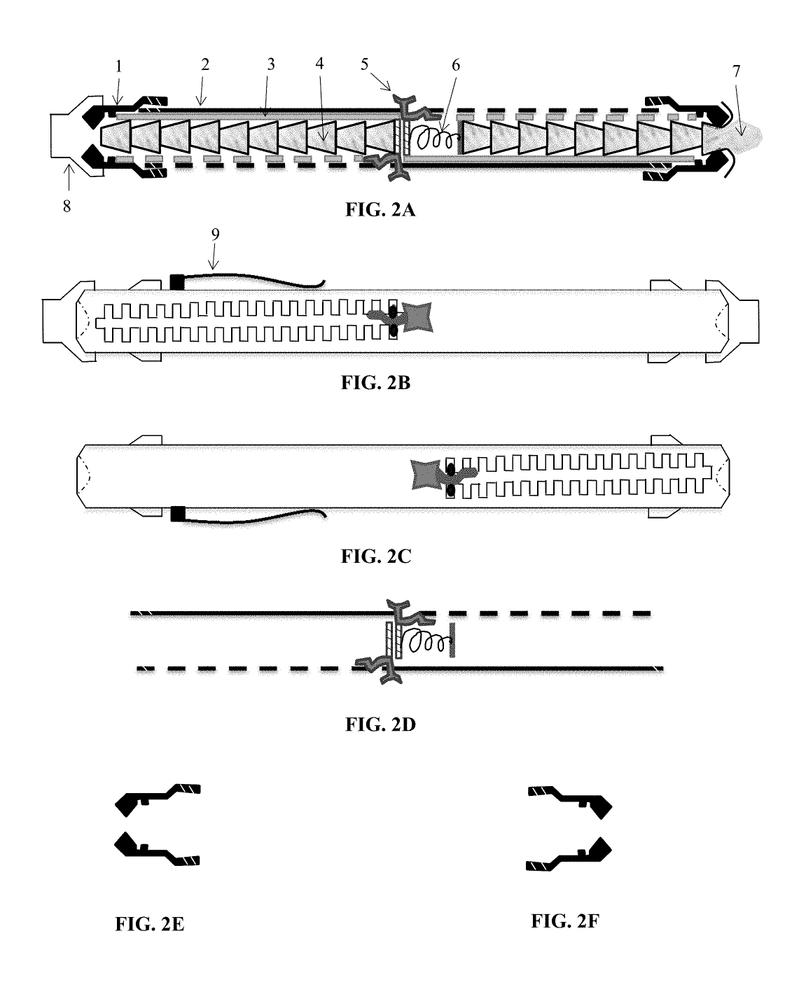

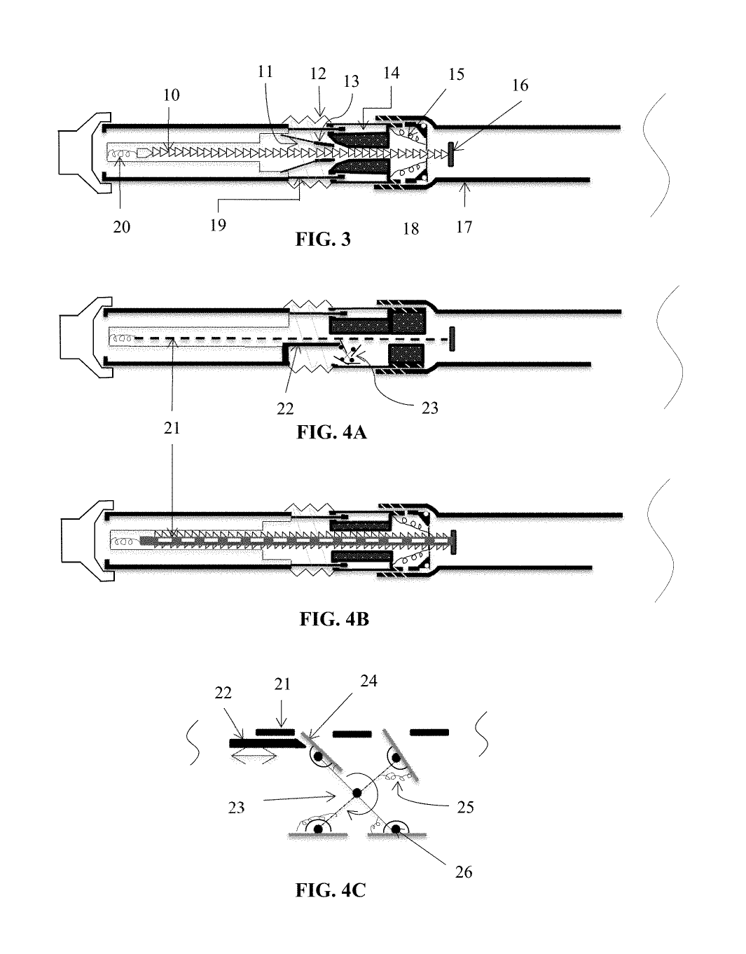

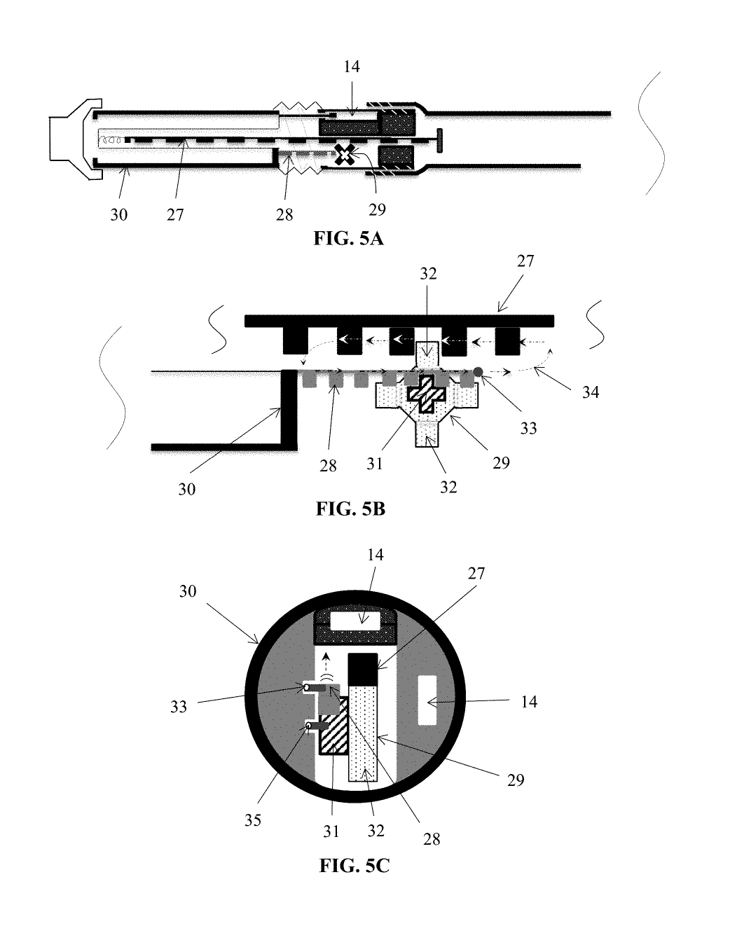

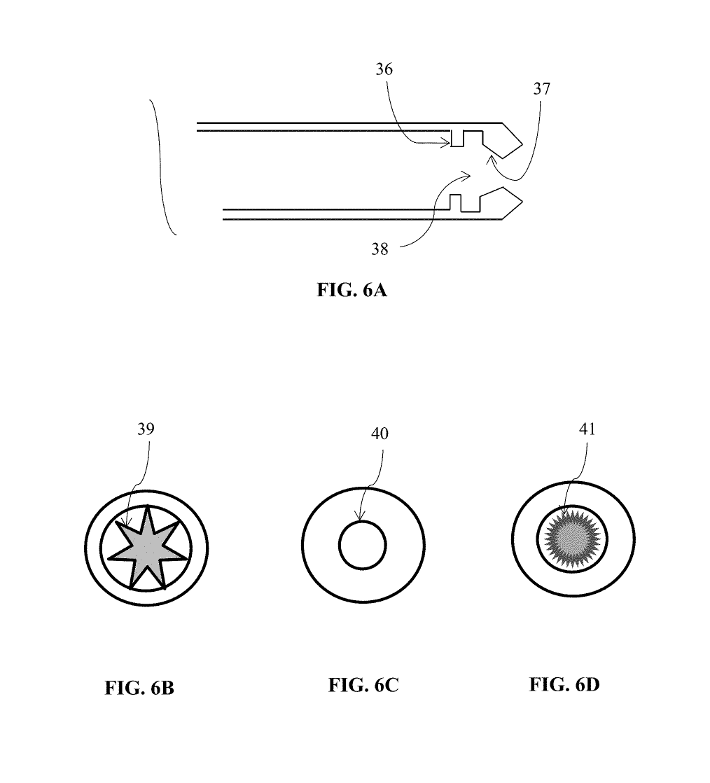

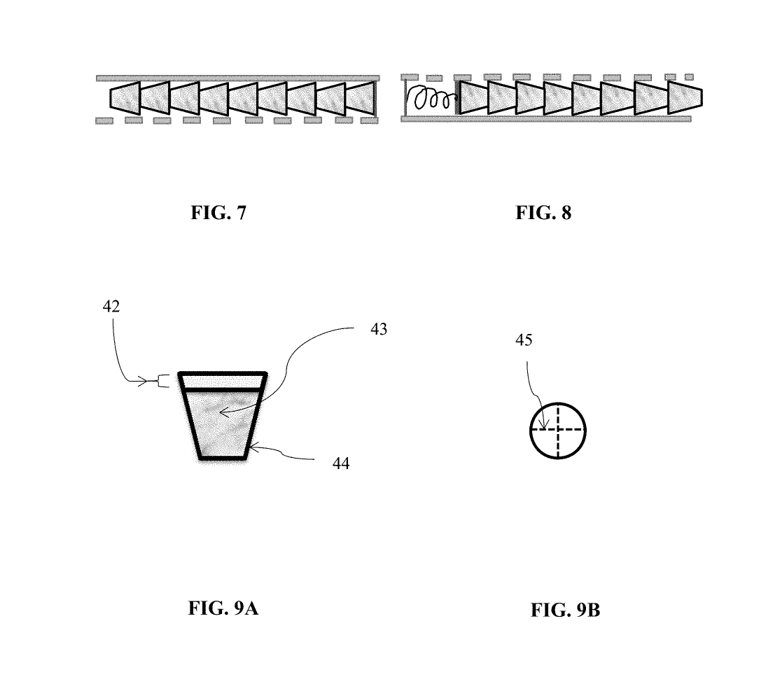

The Description of the invention is grouped into four parts. The first part is several variants of an outer cylindrical or hollow body that houses inner cylindrical refill packed with one or more refill Pellet. Examples are shown in FIG. 2D, FIG. 3, FIG. 4 and FIG. 5. The second part is several variants of a peeling mechanism that attaches to a tip of the outer cylindrical body. An example is shown in FIG. 6. The third part is several variants of inner cylindrical or hollow body that houses one or more Pellets. An example is shown in FIG. 7 and FIG. 8. The fourth part is several variants of one or more Pellet. An example of which is shown in FIG. 9.

Combination of the first part and second peeling part together with the refill package of the third and fourth parts form one apparatus to invent the “Alcohol Pen” or “Prep Pad Pen” and its variants.

FIG. 2A shows an example of a complete apparatus. It has an outer cylindrical body with a pushing lever, two inner refill cylindrical bodies stacked with encapsulated Pellets, a screw on peeling mechanism on each end, and a cap. FIG. 2E and FIG. 2F show the detached left and right peeling mechanism respectively. The peeling mechanism 1 is attached to an outer cylindrical body 2. The outer cylindrical body has a bay for 3 refill of inner cylindrical body. The inner cylindrical body in turn houses stacked up 4 refill Pellets. The apparatus allows two independent inner cylindrical refill bodies. This allows combination of refills. For example, one refill bay may contain Pellet soaked with alcohol antiseptic or antibacterial solution while the other refill bay may contain dry but sterile cotton Pellet. A 5 Pushing lever mechanism may or may not have a 6 spring attached to it. The 6 spring gives comfort and ease in pushing out refill Pellets. A Pellet 7 is squeezed out being unwrapped or peeled in the process. The widening geometry of the Pellet combined with a narrowing channel in the peeling mechanism peels the Pellets wrap or capsule as the pushing mechanism applies pressure. This will expose the Pellet and gets it ready for use, typically for one time application. The apparatus may be covered by a 8 cap at each end of the outer cylindrical body. It may also be kept in a pocket using a 9 pocket clip for ease of carrying.

FIG. 3 shows another version of a pushing mechanism. The top half of the outer cylindrical body is pushed down, in the process pushing a 10 rod with a cone shaped edge. The rod is pinched via a 13 pincer like levers as it gets guided through a narrowing channel. The top half of the outer cylindrical body squeezes down into a 14 hollow cavity guided by a 19 gliding edge. On every downward push, the rod slips through an 18 ‘L’ shaped lever and moves out a 16 pushing plate. The lever prevents the rod from going back up as a 15 lever spring locks it back in place and 17 bottom part of the screwed in outer cylindrical body prevents it from rotating back too much. Note that when a refill is loaded by unscrewing the 17 bottom half, the lever spring 15 rotates the lever far enough to allow a pull spring 20 to retract the pushing rod all the way back up. With the bottom half 17 outer cylindrical body screwed on, after every push down, an 11 outer body spring covered by rubber 12 pushes back the top half of the outer cylindrical body with enough force to free up the pincer like grasp of lever 13. This gets the push down mechanism ready for a second push.

FIG. 4A, FIG. 4B and FIG. 4C shows the first of two rotating pushing mechanism. Here the pushing rod 21 is flat with some thickness. It has rectangular holes on one side and a triangular edge on the other side. A 22 different form of lever pushes down a 23 rotating type mechanism with 24 hinged flaps and an anchoring spring 25 on the inner end. When the 22 lever pushes down on the 24 hinged flap, the flap rotates and enters into a rectangular hole of the pushing rod, 21 pushing further end plate 16. The inner spring 25 keeps the lever back in position when the 26 hinged flap 24 passes along the inner outside cylindrical wall. The hinge 26 also allows the lever 22 to retract back to position after each push when the outer spring 11 puts the upper outer cylindrical body back in place.

FIG. 5A, FIG. 5B and FIG. 5C shows the second of two rotating pushing mechanism. Here the pushing rod 27 is solid and geared on one side. On the other side it has a triangular edge similar to pushing rod 21 in FIG. 4B but with a solid center. When the top half of the outer cylindrical body 30 is pushed down, a geared lever 28 rotates an inner gear 31 of a one piece two teeth circular gear 29, mounted on a pivoting hinge 35. Consequently the outer teeth or gear 32 will rotate and push down the geared pushing rod 27. Once a push is complete, a protruding knob 33 gliding through a railing grove inside a roof of the outer cylindrical body guides the geared lever 28 along a return path 34 in such a way that the lever is sufficiently detached away from the inner gear 31. This allows a clear retracement to its original position. In each push or pen like click cycle the protruding knob 33 follows the railing grove path 34.

The peeling mechanism is a key part of the invention. FIG. 6A shows a cross sectional side view of the peeling mechanism. A ledge 36 allows the bottom tip of a hollow inner cylindrical refill body to rest on it. A cavity 38 collects unraveled or shriveled Pellet wrap or capsule. The cavity 38 gives way to narrowing channel 37 with a variety of peeling tip. Three sample varieties are shown in FIG. 6B, FIG. 6C and FIG. 6D. FIG. 6B shows a narrowing channel with a sharp 39 blade like edges. FIG. 6C shows a narrowing channel with a uniform constricting 40 tip. FIG. 6D shows a narrowing channel with a jagged 41 edge tip. When a capsulated or wrapped Pellet with a widening geometry is pushed through the narrowing channel the wrap or capsule sleeve is peeled off by the tip as shown in 7 FIG. 2A. The unwrapped Pellet is then ready for use.

A sample refill, a hollow inner cylindrical body is shown in FIG. 7 and FIG. 8. A stacked Pellet can be packed into a housing refill body. The hollow cylindrical body can come with a variety of hollow geometry and wall thickness.

To clarify the meaning of a Pellet, it has been defined in the “Definitions for Words in Claims”section. A particular type of Pellet is shown in FIG. 9. This subset has a hard material 42 anchoring absorbing material 43 that may or may not be soaked with a variety of solutions. In this particular case the Pellet is wrapped or capsulated 44 with one or more layers of liquid tight and vapor tight material. The bottom of a Pellet may or may not be perforated 45 to establish a weak place that can be torn in the peeling process.

### Pen Like Outer Cylindrical Instrument

1. The invention claim includes but not limited to a device used in Automatic Peeling which exhibits a narrowing channel at its tip that tears a wrapped or encapsulated Pellet as it passes through its channel, an example of which is shown in FIG. 6.

2. The invention claim includes but not limited to a cylindrical body with an embedded rod or plate that actively pushes down every pen like click using a non-gravity based pincer like pushing mechanism as shown in FIG. 3, or a non-gravity based rotating pushing mechanism of which one type rotates a gear with a collapsing hinge to allow retracement to original position as shown in FIG. 4 or a second type that rotates down a gear every click and retraces back using a guide that disconnects a lever from the rotating gear as shown in FIG. 5.
Inner Cylindrical or Hollow Refill or One Time Use

3. The invention claim includes but not limited to the use of a cylindrical or hollow body to house one or more Pellet in a way that can be used to directly or indirectly disinfect, cleanse or prepare, i) a living body surface prior to or post injection, incision, intrusion, insertion or application of any foreign body or, ii) a non-living body surface such as electronics, lenses, cameras, medical or non-medical devices or beauty products.
